(19) United States
(12) Patent Application Publication
(10) Pub. No.: US 2022/0193424 A1
(43) Pub. Date: Jun. 23, 2022

(54) ELECTRICAL CONTACT AND SEAL INTERCONNECT ASSEMBLY FOR IMPLANTABLE PULSE GENERATOR

(71) Applicant: Advanced Neuromodulation Systems, Inc., Plano, TX (US)

(72) Inventor: Syed Askari-Raza Baqar, Frisco, TX (US)

(21) Appl. No.: 17/214,499

(22) Filed: Mar. 26, 2021

Related U.S. Application Data
(60) Provisional application No. 63/128,656, filed on Dec. 21, 2020.

(51) Int. Cl. A61N 1/375; A61N 1/36

(52) U.S. Cl. CPC A61N 1/3754; A61N 1/36175

(57) ABSTRACT

An electrical connector for an implantable medical device includes a connector member having a substantially cylindrical outer body and a ring shaped electrical connector housed within a central opening of the outer body and an annular channel formed on a first face of the cylindrical outer body. A substantially cylindrical seal member includes an annular shoulder formed on a first face thereof. The annular shoulder is configured to seat within the annular channel of the outer body with one or more of an interference fit or a snap-fit.

### PRIORITY

This application claims priority to U.S. Provisional Application No. 63/128,656, filed Dec. 21, 2020, the contents of which are hereby incorporated by reference in its entirety.

### FIELD

The present disclosure relates generally to a connector assembly for an implantable pulse generator for accepting one or more electrical connections for stimulation leads. More particularly, the present disclosure relates to sealing and alignment devices for the connector assembly.

### BACKGROUND OF THE INVENTION

Neurostimulation systems are devices that generate electrical pulses and deliver the pulses to neural tissue of a patient to treat a variety of disorders. One category of neurostimulation systems is deep brain stimulation (DBS). In DBS, pulses of electrical current are delivered to target regions of a subject's brain, for example, for the treatment of movement and effective disorders such as PD and essential tremor. Another category of neurostimulation systems is spinal cord stimulation (SCS) which is often used to treat chronic pain such as Failed Back Surgery Syndrome (FBSS) and Complex Regional Pain Syndrome (CRPS). Dorsal root ganglion (DRG) stimulation is another example of a neurostimulation therapy in which electrical stimulation is provided to the dorsal root ganglion structure that is just outside of the epidural space. DRG stimulation is generally used to treat chronic pain.

Neurostimulation systems generally include a pulse generator and one or more leads. A stimulation lead includes a lead body made of insulative material that encloses wire conductors. The distal end of the stimulation lead includes multiple electrodes, or contacts, that intimately impinge upon patient tissue and are electrically coupled to the wire conductors. The proximal end of the lead body includes multiple terminals (also electrically coupled to the wire conductors) that are adapted to receive electrical pulses. In DBS systems, the distal end of the stimulation lead is implanted within the brain tissue to deliver the electrical pulses. The stimulation leads are then tunneled to another location within the patient's body to be electrically connected with a pulse generator or, alternatively, to an “extension.” The pulse generator is typically implanted in the patient within a subcutaneous pocket created during the implantation procedure.

The pulse generator is typically implemented using a metallic housing (or can) that encloses circuitry for generating the electrical stimulation pulses, control circuitry, communication circuitry, a rechargeable or primary cell battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on the proximal end of a stimulation lead.

In known pulse generators, the stimulation leads are held within the header using seals and spring connectors that provide an electrical connection and a sealing function in the header. However, in some instances the there is a misalignment between the seals and the spring housing, which may in some instances negatively affect the electrical connectivity and sealing effect. Due to misalignment between spring housing and seal, in certain cases, fluid can seep through or the insertion force could be undesirably high when a lead is inserted into the bore of the header.

### BRIEF DESCRIPTION OF THE INVENTION

In one embodiment, an electrical connector for an implantable medical device includes a connector member having a substantially cylindrical outer body and a ring shaped electrical connector housed within a central opening of the outer body and an annular channel formed on a first face of the cylindrical outer body. A substantially cylindrical seal member includes an annular shoulder formed on a first face thereof. The annular shoulder is configured to seat within the annular channel of the outer body with one or more of an interference fit or a snap-fit.

In another embodiment, an implantable pulse generator for generation of electrical pulses for application to tissue of a patient includes: a housing component containing electrical circuitry for generating the electrical pulses; a header component connected to the housing component, wherein the header component comprises at least one bore for receiving a stimulation lead for applying the electrical pulses to the tissue of the patient; wherein the bore comprises a plurality of connector members and cylindrical seal members arranged in an alternating sequence, wherein each of the connector members comprises a substantially cylindrical outer body and a ring shaped electrical connector housed within a central opening of the outer body and an annular channel formed on a first face of the cylindrical outer body; and wherein each of the cylindrical seal members comprises an annular shoulder formed on a first face thereof, the annular shoulder is configured to seat within the annular channel of the of the outer body with one or more of an interference fit or a snap-fit.

### BRIEF DESCRIPTION OF THE DRAWINGS

FIG. 1 is a schematic view of a stimulation system according to an embodiment of the present disclosure.

FIG. 2 is a schematic view of an embodiment of a computing device of a stimulation device of the present disclosure.

FIG. 3 is a schematic view of an embodiment of a network environment for remote management of patient care according to the present disclosure.

FIG. 4 is a profile view of an exemplary implantable stimulation device with a portion of a sidewall of the header removed to provide a view of components therein.

FIG. 5 is a cross-sectional view of an exemplary connector block of the present disclosure.

FIG. 6 is a perspective view of an electrical connector and seal according to an embodiment of the present disclosure.

FIGS. 7A and 7B shows a detail section view of an annular shoulder and channel of the electrical connector and seal of FIG. 6.

### DETAILED DESCRIPTION OF THE INVENTION

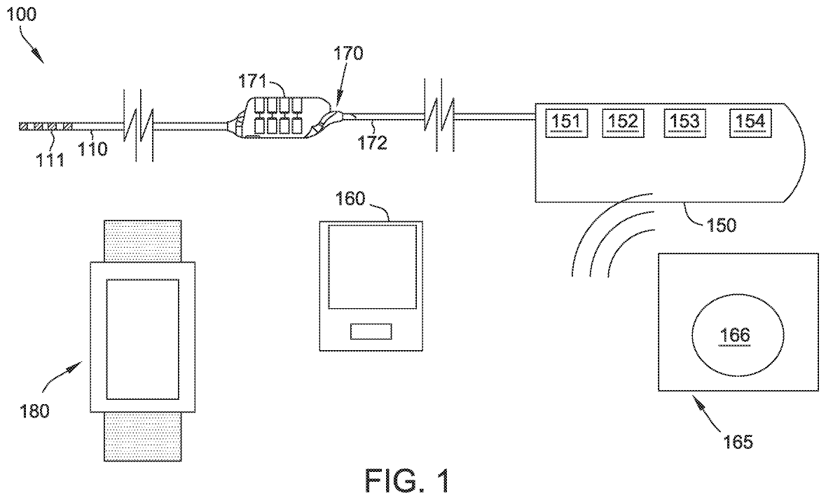

A stimulation system 100 is generally shown in FIG. 1 according to some embodiments. Stimulation system 100 generates electrical pulses for application to tissue of a patient to treat one or more disorders of the patient. System 100 includes an implantable pulse generator (IPG) 150 that is adapted to generate electrical pulses for application to tissue of a patient. Examples of commercially available implantable pulse generators include the PROCLAIM XR™ and INFINITY™ implantable pulse generators (available from ABBOTT, PLANO TX). Alternatively, in some embodiments, system 100 may include an external pulse generator (EPG) positioned outside the patient's body. IPG 150 typically includes a metallic housing (or “can”) that encloses a controller 151, pulse generating circuitry 152, a battery 153, far-field and/or near field communication circuitry 154 (e.g., BLUETOOTH communication circuitry), and other appropriate circuitry and components of the device. Controller 151 typically includes a microcontroller or other suitable processor for controlling the various other components of the device. Software code is typically stored in memory of IPG 150 for execution by the microcontroller or processor to control the various components of the device.

IPG 150 may comprise one or more attached extension components 170 or be connected to one or more separate extension components 170. Alternatively, one or more stimulation leads 110 may be connected directly to IPG 150. Within IPG 150, electrical pulses are generated by pulse generating circuitry 152 and are provided to switching circuitry. The switching circuit connects to output wires, metal ribbons, traces, lines, or the like (not shown) from the internal circuitry of pulse generator 150 to output connectors (not shown) of pulse generator 150 which are typically contained in the “header” structure of pulse generator 150. Commercially available ring/spring electrical connectors are frequently employed for output connectors of pulse generators (e.g., “Bal-Seal” brand connectors). The terminals of one or more stimulation leads 110 are inserted within connector portion 171 for electrical connection with respective connectors or directly within the header structure of pulse generator 150. Thereby, the pulses originating from IPG 150 are conducted to electrodes 111 through wires contained within the lead body of lead 110. The electrical pulses are applied to tissue of a patient via electrodes 111.

For implementation of the components within IPG 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference in its entirety. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference in its entirety.

An example and discussion of “constant current” pulse generating circuitry is provided in U.S. Patent Publication No. 2006/0170486 entitled “PULSE GENERATOR HAVING AN EFFICIENT FRACTIONAL VOLTAGE CONVERTER AND METHOD OF USE,” which is incorporated herein by reference in its entirety. One or multiple sets of such circuitry may be provided within IPG 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number and type of electrodes 111, terminals, and internal conductors.

External controller device 160 is a device that permits the operations of IPG 150 to be controlled by a user after IPG 150 is implanted within a patient. Also, multiple controller devices 160 may be provided for different types of users (e.g., the patient or a clinician). Controller device 160 can be implemented by utilizing a suitable handheld processor-based system that possesses wireless communication capabilities. In some embodiments, controller device 160 may be a smart phone or mobile electronic device configured to operate as controller device 160 described herein. Software is typically stored in a nontransitory memory of controller device 160 to control the various operations of controller device 160. The interface functionality of controller device 160 is implemented using suitable software code for interacting with the user and using the wireless communication capabilities to conduct communications with IPG 150. One or more user interface display screens may be provided in software to allow the patient and/or the patient's clinician to control operations of IPG 150 using controller device 160. In some embodiments, commercially available devices such as APPLE IOS devices are adapted for use as controller device 160 by include one or more “apps” that communicate with IPG 150 using, for example, BLUETOOTH® or other short range wireless communication systems.

Controller device 160 preferably provides one or more user interfaces to allow the user to operate IPG 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc.

Controller device 160 may permit programming of IPG 150 to provide a number of different stimulation patterns or therapies to the patient as appropriate for a given patient and/or disorder. Examples of different stimulation therapies include conventional tonic stimulation (continuous train of stimulation pulses at a fixed rate), BurstDR stimulation (burst of pulses repeated at a high rate interspersed with quiescent periods with or without duty cycling), “high frequency” stimulation (e.g., a continuous train of stimulation pulses at 10,000 Hz), noise stimulation (series of stimulation pulses with randomized pulse characteristics such as pulse amplitude to achieve a desired frequency domain profile). Any suitable stimulation pattern or combination thereof can be provided by IPG 150 according to some embodiments. Controller device 160 communicates the stimulation parameters and/or a series of pulse characteristics defining the pulse series to be applied to the patient to IPG 150 to generate the desired stimulation therapy.

Examples of suitable therapies include tonic stimulation (in which a fixed frequency pulse train) is generated, burst stimulation (in which bursts of multiple high frequency pulses) are generated which in turn are separated by quiescent periods, “high frequency” stimulation, multi-frequency stimulation, noise stimulation. Descriptions of respective neurostimulation therapies are provided in the following publications: (1) Schu S., Slotty P. J., Bara G., von Knop M., Edgar D., Vesper J. A Prospective, Randomised, Double-blind, Placebo-controlled Study to Examine the Effectiveness of Burst Spinal Cord Stimulation Patterns for the Treatment of Failed Back Surgery Syndrome. Neuromodulation 2014; 17: 443-450; (2) Al-Kaisy Al, Van Buyten JP, Smet I, Palmisani S, Pang D, Smith T. 2014. Sustained effectiveness of 10 kHz high-frequency spinal cord stimulation for patients with chronic, low back pain: 24-month results of a prospective multicenter study. Pain Med. 2014 March; 15(3):347-54; and (3) Sweet, Badjatiya, Tan D1, Miller. Paresthesia-Free High-Density Spinal Cord Stimulation for Postlaminectomy Syndrome in a Prescreened Population: A Prospective Case Series. Neuromodulation. 2016 April; 19(3):260-7. Noise stimulation is described in U.S. Patent No. U.S. Pat. No. 8,682,441B2. Burst stimulation is described in U.S. Pat. No. 8,224,453 and U.S. Published Application No. 20060095088. All of these references are incorporated herein by reference in their entireties.

In one embodiment, for implementation of the components within stimulation system 100, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference in its entirety. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION” which is incorporated herein by reference in its entirety.

In one embodiment, IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 2001/093953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference in their entireties.

External charger device 165 may be provided to recharge battery 153 of IPG 150 according to some embodiments when IPG 150 includes a rechargeable battery. External charger device 165 comprises a power source and electrical circuitry (not shown) to drive current through coil 166. The patient places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. In operation during a charging session, external charger device 165 generates an AC-signal to drive current through coil 166 at a suitable frequency. Assuming that primary coil 166 and secondary coil are suitably positioned relative to each other, the secondary coil is disposed within the magnetic field generated by the current driven through primary coil 166. Current is then induced by a magnetic field in the secondary coil. The current induced in the coil of the implantable pulse generator is rectified and regulated to recharge the battery of IPG 150. IPG 150 may also communicate status messages to external charging device 165 during charging operations to control charging operations. For example, IPG 150 may communicate the coupling status, charging status, charge completion status, etc.

System 100 may include external wearable device 180 such as a smartwatch or health monitor device. Wearable device may be implemented using commercially available devices such as FITBIT VERSA SMARTWATCH™, SAMSUNG GALAXY SMARTWATCH™, and APPLE WATCH™ devices with one or more apps or appropriate software to interact with IPG 150 and/or controller device 160. In some embodiments, wearable device 180, controller device 160, and IPG 150 conduct communications using BLUETOOTH® communications.

Wearable device 180 monitors activities of the patient and/or senses physiological signals. Wearable device 180 may track physical activity and/or patient movement through accelerometers. Wearable device 180 may monitory body temperature, heart rate, electrocardiogram activity, blood oxygen saturation, and/or the like. Wearable device 180 may monitor sleep quality or any other relevant health related activity.

Wearable device 180 may provide one or more user interface screens to permit the patient to control or otherwise interact with IPG 150. For example, the patient may increase or decrease stimulation amplitude, change stimulation programs, turn stimulation on or off, and/or the like using wearable device 180. Also, the patient may check the battery status of other implant status information using wearable device 180.

Wearable device 180 may include one or more interface screens to receive patient input. In some embodiments, wearable device 180 and/or controller device 160 are implemented (individually or in combination) to provide an electronic patient diary function. The patient diary function permits the patient to record on an ongoing basis the health status of the patient and the effectiveness of the therapy for the patient. In some embodiments as discussed herein, wearable device 180 and/or controller device 160 enable the user to indicate the current activity of the patient, the beginning of an activity, the completion of an activity, the ease or quality of patient's experience with a specific activity, the patient's experience of pain, the patient's experience of relief from pain by the stimulation, or any other relevant indication of patient health by the patient.

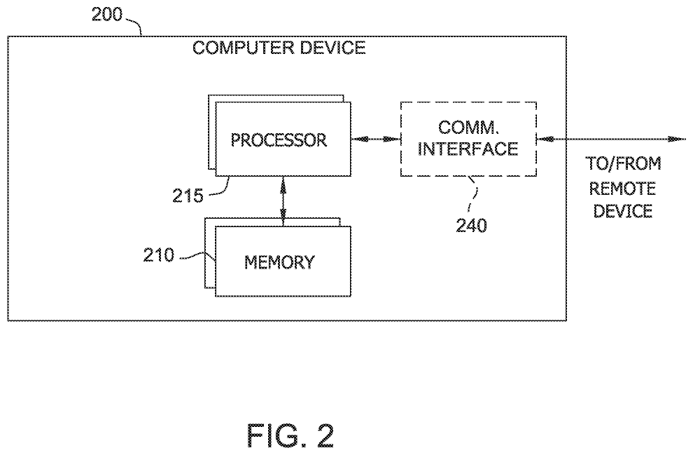

FIG. 2 is a block diagram of one embodiment of a computing device 200 that may be used to according to some embodiments. Computing device 200 may be used to implement external controller device 160, wearable device 180, remote care management servers, or other computing system according to some embodiments.

Computing device 200 includes at least one memory device 210 and a processor 215 that is coupled to memory device 210 for executing instructions. In some embodiments, executable instructions are stored in memory device 210, which may comprise a nontransitory memory. In some embodiments, computing device 200 performs one or more operations described herein by programming processor 215. For example, processor 215 may be programmed by encoding an operation as one or more executable instructions and by providing the executable instructions in memory device 210.

Processor 215 may include one or more processing units (e.g., in a multi-core configuration). Further, processor 215 may be implemented using one or more heterogeneous processor systems in which a main processor is present with secondary processors on a single chip. In another illustrative example, processor 215 may be a symmetric multi-processor system containing multiple processors of the same type. Further, processor 215 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein.

In the illustrated embodiment, memory device 210 is one or more devices that enable information such as executable instructions and/or other data to be stored and retrieved. Memory device 210 may include one or more computer readable media, such as, without limitation, dynamic random access memory (DRAM), read-only memory (ROM), electrically erasable programmable read-only memory (EEPROM), static random access memory (SRAM), a solid state disk, and/or a hard disk. Memory device 210 may be configured to store, without limitation, application source code, application object code, source code portions of interest, object code portions of interest, configuration data, execution events and/or any other type of data.

Computing device 200, in the illustrated embodiment, includes a communication interface 240 coupled to processor 215. Communication interface 240 communicates with one or more remote devices, such as a clinician or patient programmer. To communicate with remote devices, communication interface 240 may include, for example, a wired network adapter, a wireless network adapter, a radio-frequency (RF) adapter, and/or a mobile telecommunications adapter.

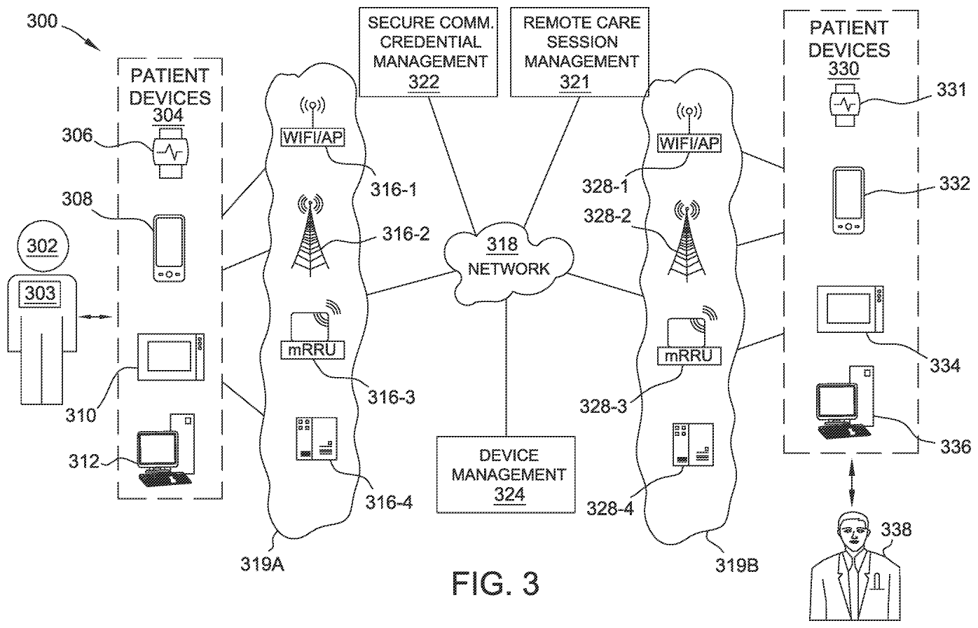

FIG. 3 depicts a network environment 300 for remote management of patient care. One or more embodiments of a remote care therapy application or service may be implemented in network environment 300, as described herein. In general, “remote care therapy” may involve any care, biomedical monitoring, or therapy that may be provided by a clinician, a medical professional or a healthcare provider, and/or their respective authorized agents (including digital/virtual assistants), with respect to a patient over a communications network while the patient and the clinician/provider are not in close proximity to each other (e.g., not engaged in an in-person office visit or consultation). Accordingly, in some embodiments, a remote care therapy application may form a telemedicine or a telehealth application or service that not only allows healthcare professionals to use electronic communications to evaluate, diagnose and treat patients remotely, thereby facilitating efficiency as well as scalability, but also provides patients with relatively quick and convenient access to diversified medical expertise that may be geographically distributed over large areas or regions, via secure communications channels as described herein.

Network environment 300 may include any combination or sub-combination of a public packet-switched network infrastructure (e.g., the Internet or worldwide web, also sometimes referred to as the “cloud”), private packet-switched network infrastructures such as Intranets and enterprise networks, health service provider network infrastructures, and the like, any of which may span or involve a variety of access networks, backhaul and core networks in an end-to-end network architecture arrangement between one or more patients, e.g., patient(s) 302, and one or more authorized clinicians, healthcare professionals, or agents thereof, e.g., generally represented as caregiver(s) or clinician(s) 338.

Example patient(s) 302, each having a suitable implantable device 303, may be provided with a variety of corresponding external devices for controlling, programming, otherwise (re)configuring the functionality of respective implantable medical device(s) 303, as is known in the art. Such external devices associated with patient(s) 302 are referred to herein as patient devices 304, and may include a variety of user equipment (UE) devices, tethered or untethered, that may be configured to engage in remote care therapy sessions. By way of example, patient devices 304 may include smartphones, tablets or phablets, laptops/desktops, handheld/palmtop computers, wearable devices such as smart glasses and smart watches, personal digital assistant (PDA) devices, smart digital assistant devices, etc., any of which may operate in association with one or more virtual assistants, smart home/office appliances, smart TVs, virtual reality (VR), mixed reality (MR) or augmented reality (AR) devices, and the like, which are generally exemplified by wearable device(s) 306, smartphone(s) 308, tablet(s)/phablet(s) 310 and computer(s) 312. As such, patient devices 304 may include various types of communications circuitry or interfaces to effectuate wired or wireless communications, short-range and long-range radio frequency (RF) communications, magnetic field communications, Bluetooth communications, etc., using any combination of technologies, protocols, and the like, with external networked elements and/or respective implantable medical devices 303 corresponding to patient(s) 302.

With respect to networked communications, patient devices 304 may be configured, independently or in association with one or more digital/virtual assistants, smart home/premises appliances and/or home networks, to effectuate mobile communications using technologies such as Global System for Mobile Communications (GSM) radio access network (GRAN) technology, Enhanced Data Rates for Global System for Mobile Communications (GSM) Evolution (EDGE) network (GERAN) technology, 4G Long Term Evolution (LTE) technology, Fixed Wireless technology, 5th Generation Partnership Project (5GPP or 5G) technology, Integrated Digital Enhanced Network (IDEN) technology, WiMAX technology, various flavors of Code Division Multiple Access (CDMA) technology, heterogeneous access network technology, Universal Mobile Telecommunications System (UMTS) technology, Universal Terrestrial Radio Access Network (UTRAN) technology, All-IP Next Generation Network (NGN) technology, as well as technologies based on various flavors of IEEE 802.11 protocols (e.g., WiFi), and other access point (AP)-based technologies and microcell-based technologies such as femtocells, picocells, etc. Further, some embodiments of patient devices 104 may also include interface circuitry for effectuating network connectivity via satellite communications. Where tethered UE devices are provided as patient devices 304, networked communications may also involve broadband edge network infrastructures based on various flavors of Digital Subscriber Line (DSL) architectures and/or Data Over Cable Service Interface Specification (DOCSIS)-compliant Cable Modem Termination System (CMTS) network architectures (e.g., involving hybrid fiber-coaxial (HFC) physical connectivity). Accordingly, by way of illustration, an edge/access network portion 119A is exemplified with elements such as WiFi/AP node(s) 316-1, macro/microcell node(s) 116-2 and 116-3 (e.g., including micro remote radio units or RRUs, base stations, eNB nodes, etc.) and DSL/CMTS node(s) 316-4.

Similarly, clinicians 338 may be provided with a variety of external devices for controlling, programming, otherwise (re)configuring or providing therapy operations with respect to one or more patients 302 mediated via respective implantable medical device(s) 303, in a local therapy session and/or remote therapy session, depending on implementation and use case scenarios. External devices associated with clinicians 338, referred to herein as clinician devices 330, may include a variety of UE devices, tethered or untethered, similar to patient devices 304, which may be configured to engage in remote care therapy sessions as will be set forth in detail further below. Clinician devices 330 may therefore also include devices (which may operate in association with one or more virtual assistants, smart home/office appliances, VRAR virtual reality (VR) or augmented reality (AR) devices, and the like), generally exemplified by wearable device(s) 331, smartphone(s) 332, tablet(s)/phablet(s) 334 and computer(s) 336. Further, example clinician devices 330 may also include various types of network communications circuitry or interfaces similar to that of patient device 304, which may be configured to operate with a broad range of technologies as set forth above. Accordingly, an edge/access network portion 319B is exemplified as having elements such as WiFi/AP node(s) 328-1, macro/microcell node(s) 328-2 and 328-3 (e.g., including micro remote radio units or RRUs, base stations, eNB nodes, etc.) and DSL/CMTS node(s) 328-4. It should therefore be appreciated that edge/access network portions 319A, 319B may include all or any subset of wireless communication means, technologies and protocols for effectuating data communications with respect to an example embodiment of the systems and methods described herein.

In one arrangement, a plurality of network elements or nodes may be provided for facilitating a remote care therapy service involving one or more clinicians 338 and one or more patients 302, wherein such elements are hosted or otherwise operated by various stakeholders in a service deployment scenario depending on implementation (e.g., including one or more public clouds, private clouds, or any combination thereof). In one embodiment, a remote care session management node 320 is provided, and may be disposed as a cloud-based element coupled to network 318, that is operative in association with a secure communications credentials management node 322 and a device management node 324, to effectuate a trust-based communications overlay/tunneled infrastructure in network environment 300 whereby a clinician may advantageously engage in a remote care therapy session with a patient.

In the embodiments described herein, implantable medical device 303 may be any suitable medical device. For example, implantable medical device may be a neurostimulation device that generates electrical pulses and delivers the pulses to nervous tissue of a patient to treat a variety of disorders.

Although implantable medical device 303 is described in the context of a neurostimulation device herein, those of skill in the art will appreciate that implantable medical device 303 may be any type of implantable medical device.

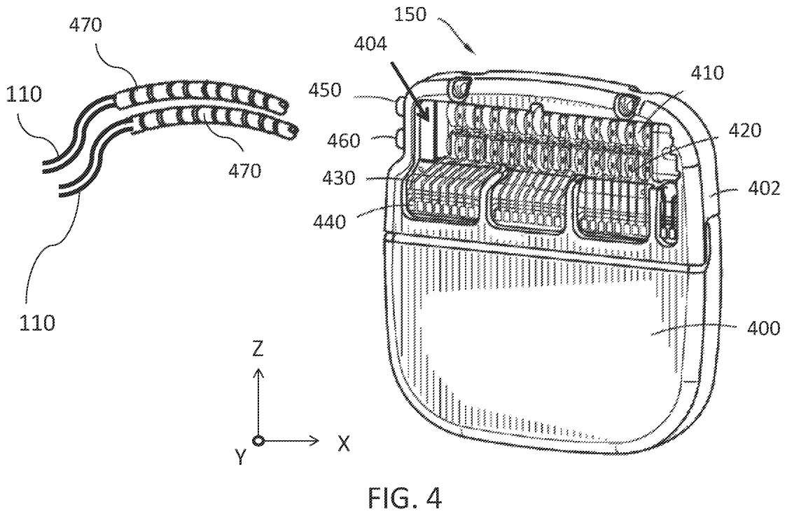

With reference to FIG. 4, in one embodiment, the implantable pulse generator 150 comprises a main body 400 and a header 402. A portion of the header 402 is shown as removed to provide a view of the components therein. In one embodiment, two sets of electrical connectors, connectors 410 and 420 are enclosed within the header 402. In some embodiments, the main body 400 is metallic, but in other embodiments main body 400 may comprise other biocompatible materials such as plastic or the like. In one embodiment, the header 402 is removably securable to main body 400. The main body 400 is a housing that houses controller 151, pulse generating circuitry 152, a battery 153, far-field and/or near field communication circuitry 154 (e.g., BLUETOOTH communication circuitry), and other appropriate circuitry and components of the device connector assembly (FIG. 1). In one embodiment, the header 402 includes a connector block 404 therein.

The connector 410 defines the bore 450, and the connector 420 defines the bore 460. A terminal 470 of a stimulation lead 110 is receivable in the bore 450 or bore 460. Although two connectors 410 and 420 are shown in FIG. 4, in other embodiments any number of connectors may be used that allows the device to operate as described herein, such as from 1 connector to 10 or more connectors for receiving additional terminals 470 of corresponding stimulation leads 110. In one embodiment, the connectors 410, 420 each include a stack of contact blocks separated by seals. In one embodiment, the contact blocks in connectors 410, 420 are coupled to respective lead frames 430. In one embodiment, the lead frames 430 may extend from the contact blocks and be coupled to respective feed through pins 440, which may be housed in one or more ceramic blocks or other suitable materials. Each terminal 470 of the stimulation lead can have substantially any diameter. By way of example, the diameter of the terminal or the stimulation lead 110 can be from 0.025 to 0.1 inches, such as 0.050 inches, or any other diameter that allows the devices to operate as described herein. Correspondingly, the bores 600, 604 of the connector and seal may have the same or similar diameters.

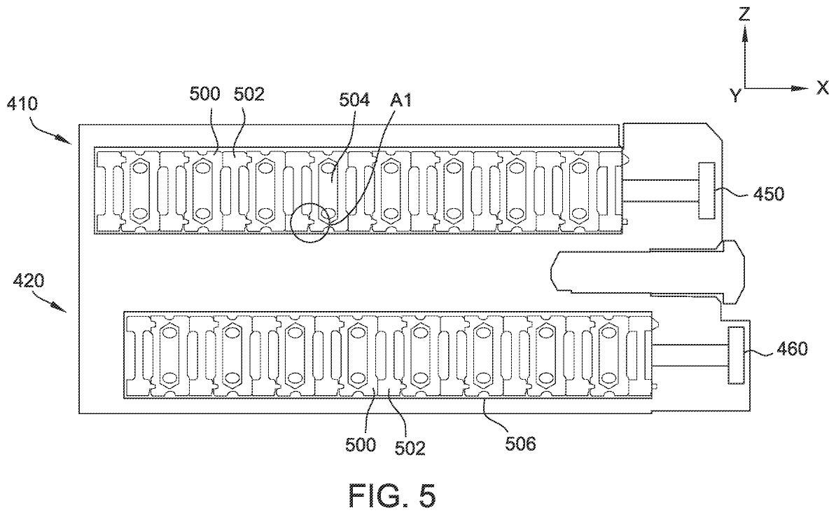

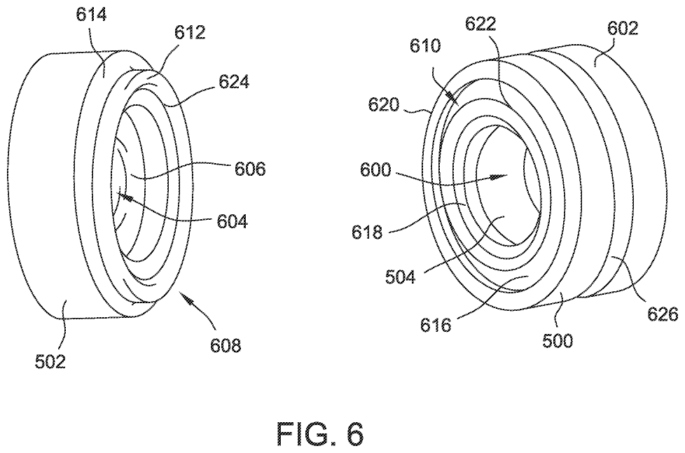

A cross-section of the header 402 taken along the X-Z plane is shown in FIG. 5. As shown, each of bores 450 and 460 comprise a plurality of connectors 500 separated by a plurality of seals 502. With additional reference to FIG. 6, the connectors and seals are described. FIG. 6 shows a profile view of an embodiment of a seal 502 and a connector 500 of the present disclosure. In one embodiment, the connectors 500 are generally cylindrical in shape and comprise an outer ring 602 and an electrical connector 504 having a through bore 600 therethrough. In one embodiment, the electrical connectors 504 may be, for example ring shaped terminals such as Bal-Seal® connectors or the like. In some embodiments, the electrical connector 504 is a conductive spring configured to apply a compressive force to a terminal 470 inserted therein. The electrical connectors 504 comprise an electrically conductive material, such as metal, metal alloys or the like, such as titanium, stainless steel, pyrolytic carbon, zirconium, niobium, molybdenum, palladium, hafnium, tantalum, tungsten, iridium, platinum, gold, nickel, chromium, or alloys thereof. The outer ring 602 may comprise an electrically conductive material that is the same or different from electrical connectors 504. In other embodiments, the outer ring 602 is formed at least partially of an electrically non-conductive material such as plastic, rubber, polymers, urethane, silicone, PEEK or other materials that allow the outer ring to function as described herein. In one embodiment, the bores 450, 460 are configured (e.g., sized and shaped) to allow the terminals of the stimulation leads to be inserted therein. The terminal at the proximal end of the stimulation lead comprises a conductive material for interfacing with electrical connectors 504 of the connector block 404.

In one embodiment, seal 502 is generally cylindrical in shape and comprises an inner surface 606 that defines a bore 604 therethrough. Bore 604 has a diameter configured to allow a terminal 470 to be inserted therethrough. In some embodiments, the bore 604 has a diameter slightly smaller than the diameter of the terminal 470, such that the inner surface 606 resiliently expands to accept the terminal 470 therethrough, and sealingly engages the terminal 470. In embodiments, the seal 502 is formed of an electrically non-conductive material, such as rubber, plastic, polymer, urethane, silicone, PEEK or other polymers or other materials that allow the seal 502 to function as described herein.

In one embodiment, each of the seals 502 includes a first locking member 608 that is configured to sealingly engage with a second locking member 610 of connector 500. In the embodiment shown in FIG. 6, the first locking member comprises an annular shoulder 612. The annular shoulder 612 is a raised projection extending from face 614 of seal 502. In one embodiment, the second locking member 610 comprises an annular channel 616 that is configured to interlock with annular shoulder 612. The annular channel is defined by a first ring 618 and a second ring 620, defining the inner circumference and outer circumference of annular channel 616, respectively.

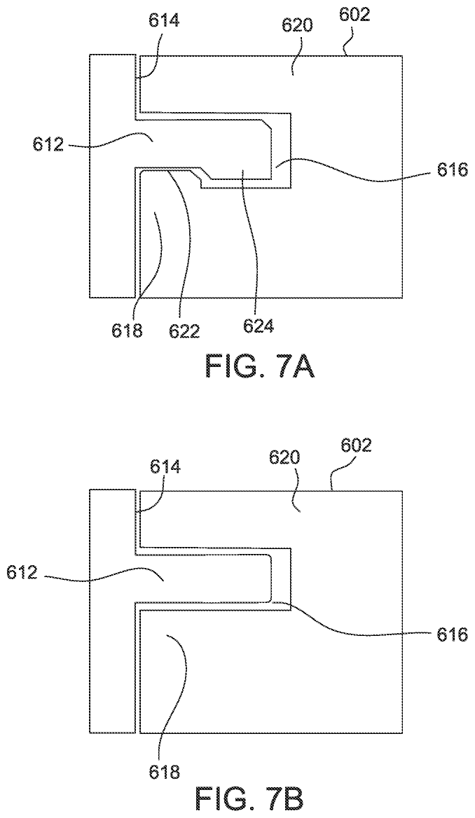

With reference to FIGS. 6 and 7A-7B, in one embodiment, the annular shoulder 612 is configured to be press-fit into annular channel 616. In some embodiments, the press-fit will provide a snap-fit, providing the user audible or physical feedback that alerts the user that the annular shoulder 612 is fully and properly seated within annular channel 616. In some embodiments, first ring 618 may comprise a lip 622 that is configured to engage a corresponding lip channel 624, when annular shoulder 612 is fully and properly seated within annular channel 616. The engagement of the lip 622 with the lip channel 624 may provide a hook like retention feature, that provides an additional holding force to maintain the seal 502 in sealing engagement with the connector 500. In other embodiments, such as shown in FIG. 7B, the annular shoulder 612 is formed without a lip channel 624 and provides an interference fit with annular channel 616 to provide the sealing and locking engagement between connector 500 and seal 502.

In embodiments, the connected seals 502 and connectors 500 are housed within the connectors 410, 420. As shown, in the exemplary embodiment shown in FIG. 5, the seals 502 and connectors 500 are seated within the connectors 410,420 in an alternating pattern. In the embodiment shown in FIG. 5, there are eight connectors 500 in each of connectors 410,420. However, in other embodiments, substantially any number of alternating connectors 500 and seals 502 may be used, such as from 1 to 20 or more depending on the desired application.

In some embodiments, the connector 500 includes a circumferential groove 626 formed into the outer ring 602 thereof. In one embodiment, the circumferential groove is sized and shaped to accept a wire lead, such as lead frames 430 for electrical interconnection with electrical connector 504. In another embodiment, the circumferential groove may engage with a circumferential projection 506 of the connectors 410, 420. Accordingly, when the circumferential projection 506 is seated to the circumferential groove 626, the connector 500 is held in position within the electrical connectors 410, 420 respectively.

In some embodiments, when the annular shoulder 612 is seated within the annular channel 616 an air tight, liquid tight or hermetic seal is formed between the seal 502 and the connector 500. The engagement of the seal 502 with the connectors 500 facilitates the pairs of seals 502/connectors 500 staying in proper position within the connectors 410, 420 that are within bores 450, 460 respectively and reducing or eliminating shifting and misalignment of the connectors 500 and seals 502.

In other embodiments, the connectors 500 may comprise a second annular groove on an opposing side of the connector 500 to the annular groove 616. The second groove may be the same or substantially similar to the annular groove 616. In this embodiment, one or more of the seals 502 may also comprise a second annular shoulder on an opposing side thereof, which may be the same or substantially similar to the annular shoulder 612. Accordingly, in this embodiment, each connector 500 may be secured to a seal 502 on each opposing side thereof by engaging the annular shoulder 612 with the annular channel 616 and the second annular shoulder with the second annular channel. In this embodiment, one or more pairs of connectors 500 and seals 502 may be coupled together to further promote stability within the connectors 410, 420.

The following embodiments are provided to illustrate aspects of the disclosure, although the embodiments are not intended to be limiting and other aspects and/or embodiments may also be provided.

Embodiment 1. An electrical connector for an implantable medical device, the electrical connector comprising: a connector member comprising a substantially cylindrical outer body and a ring shaped electrical connector housed within a central opening of the outer body and an annular channel formed on a first face of the cylindrical outer body; and a substantially cylindrical seal member comprising an annular shoulder formed on a first face thereof; wherein the annular shoulder is configured to seat within the annular channel of the of the outer body with one or more of an interference fit or a snap-fit.

Embodiment 2. The electrical connector according to Embodiment 1, wherein the first face is substantially perpendicular to a longitudinal axis of the central opening.

Embodiment 3. The electrical connector according to any preceding embodiment, wherein the ring shaped electrical connector comprises a spring connector.

Embodiment 4. The electrical connector according to any preceding embodiment, wherein the annular channel comprises a lip extending in a radial direction.

Embodiment 5. The electrical connector according to any preceding embodiment, wherein the annular shoulder of the seal member comprises a lip channel configured to engage with the lip.

Embodiment 6. The electrical connector according to any preceding embodiment, wherein the electrical connector member comprises a circumferential groove defined on an outer surface of the outer body.

Embodiment 7. The electrical connector according to any preceding embodiment, wherein the circumferential groove is configured to engage with a lead wire of a stimulation lead or a corresponding projection of a header of the implantable medical device.

Embodiment 8. The electrical connector according to any preceding embodiment, wherein the cylindrical outer body comprises a second face on an opposing side of outer body to the first face, and the second face comprises a second annular channel.

Embodiment 9. The electrical connector according to any preceding embodiment, wherein the sealing member comprises a second face on an opposing side of sealing member to the first face, and the second face comprises a second annular shoulder.

Embodiment 10. The electrical connector according to any preceding embodiment, wherein the ring shaped electrical connector comprises an electrically conductive material.

Embodiment 11. The electrical connector according to any preceding embodiment, wherein the seal member comprises an electrically insulating material.

Embodiment 12. The electrical connector according to any preceding embodiment, wherein the outer body of the electrical connector member comprises an electrically insulating or conductive material.

Embodiment 13. The electrical connector according to any preceding embodiment, wherein the ring shaped electrical connector is configured to electrically connect to a terminal of a stimulation lead.

Embodiment 14. An implantable pulse generator for generation of electrical pulses for application to tissue of a patient, comprising: a housing component containing electrical circuitry for generating the electrical pulses; a header component connected to the housing component, wherein the header component comprises at least one bore for receiving a stimulation lead for applying the electrical pulses to the tissue of the patient; wherein the bore comprises a plurality of connector members and cylindrical seal members arranged in an alternating sequence, wherein each of the connector members comprises a substantially cylindrical outer body and a ring shaped electrical connector housed within a central opening of the outer body and an annular channel formed on a first face of the cylindrical outer body; and wherein each of the cylindrical seal members comprises an annular shoulder formed on a first face thereof, the annular shoulder is configured to seat within the annular channel of the of the outer body with one or more of an interference fit or a snap-fit.

Embodiment 15. The implantable pulse generator according to embodiment 15, further comprising a stimulation lead having a terminal at one end thereof, the terminal configured to be electrically coupleable to the ring shaped electrical connector.

Embodiment 16. The implantable pulse generator according to any preceding embodiment, wherein the ring shaped electrical connector comprises a spring connector.

Embodiment 17. The implantable pulse generator according to any preceding embodiment, wherein the annular channel comprises a lip extending in a radial direction.

Embodiment 18. The implantable pulse generator according to any preceding embodiment, wherein the annular shoulder of the seal member comprises a lip channel configured to engage with the lip.

Embodiment 19. The implantable pulse generator according to any preceding embodiment, wherein the electrical connector member comprises a circumferential groove defined on an outer surface of the outer body.

Embodiment 20. The implantable pulse generator according to any preceding embodiment, wherein the circumferential groove is configured to engage with a lead wire of a stimulation lead or a corresponding projection of a header of the implantable medical device.

Embodiment 21. The implantable pulse generator according to any preceding embodiment, wherein the cylindrical outer body comprises a second face on an opposing side of outer body to the first face, and the second face comprises a second annular channel.

Embodiment 22. The implantable pulse generator according to any preceding embodiment, wherein the sealing member comprises a second face on an opposing side of sealing member to the first face, and the second face comprises a second annular shoulder.

Embodiment 23. The implantable pulse generator according to any preceding embodiment, wherein the ring shaped electrical connector comprises an electrically conductive material.

Embodiment 24. The implantable pulse generator according to any preceding embodiment, wherein the seal member comprises an electrically insulating material.

Embodiment 25. The implantable pulse generator according to any preceding embodiment, wherein the outer body of the electrical connector member comprises an electrically insulating or conductive material.

Embodiment 26. The implantable pulse generator according to any preceding embodiment, wherein the ring shaped electrical connector is configured to electrically connect to a terminal of a stimulation lead.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An electrical connector for an implantable medical device, the electrical connector comprising:
a connector member comprising a substantially cylindrical outer body and a ring shaped electrical connector housed within a central opening of the substantially cylindrical outer body and an annular channel formed on a first face of the substantially cylindrical outer body;
a substantially cylindrical seal member comprising an annular shoulder formed on a first face thereof; and
wherein the annular shoulder is configured to seat within the annular channel of the substantially cylindrical outer body with one or more of an interference fit or a snap-fit.

2. The electrical connector according to claim 1, wherein the first face is substantially perpendicular to a longitudinal axis of the central opening.

3. The electrical connector according to claim 1, wherein the ring shaped electrical connector comprises a spring connector.

4. The electrical connector according to claim 1, wherein the annular channel comprises a lip extending in a radial direction.

5. The electrical connector according to claim 4, wherein the annular shoulder of the substantially cylindrical seal member comprises a lip channel configured to engage with the lip.

6. The electrical connector according to claim 1, wherein the connector member comprises a circumferential groove defined on an outer surface of the substantially cylindrical outer body.

7. The electrical connector according to claim 6, wherein the circumferential groove is configured to engage with a lead wire of a stimulation lead.

8. The electrical connector according to claim 1, wherein the cylindrical outer body comprises a second face on an opposing side of outer body to the first face, and the second face comprises a second annular channel.

9. The electrical connector according to claim 8, wherein the substantially cylindrical seal member comprises a second face on an opposing side of sealing member to the first face, and the second face comprises a second annular shoulder.

10. The electrical connector according to claim 1, wherein the ring shaped electrical connector comprises an electrically conductive material.

11. The electrical connector according to claim 1, wherein the substantially cylindrical seal member comprises an electrically insulating material.

12. The electrical connector according to claim 1, wherein the substantially cylindrical outer body of the connector member comprises an electrically conductive material.

13. The electrical connector according to claim 1, wherein the ring shaped electrical connector is configured to electrically connect to a terminal of a stimulation lead.

14. An implantable pulse generator for generation of electrical pulses for application to tissue of a patient, comprising:
a housing component containing electrical circuitry for generating the electrical pulses;
a header component connected to the housing component, wherein the header component comprises at least one bore for receiving a stimulation lead for applying to the tissue of the patient;
wherein the bore comprises a plurality of connector members and cylindrical seal members arranged in an alternating sequence, wherein each of the connector members comprises a substantially cylindrical outer body and a ring shaped electrical connector housed within a central opening of the substantially cylindrical outer body and an annular channel formed on a first face of the cylindrical outer body; and
wherein each of the cylindrical seal members comprises an annular shoulder formed on a first face thereof, the annular shoulder is configured to seat within the annular channel of the substantially cylindrical outer body with one or more of an interference fit or a snap-fit.

15. The implantable pulse generator according to claim 14, further comprising a stimulation lead having a terminal at one end thereof, the terminal configured to be electrically coupleable to the ring shaped electrical connector.

16. The implantable pulse generator according to claim 14, wherein the ring shaped electrical connector comprises a spring connector.

17. The implantable pulse generator according to claim 14, wherein the annular channel comprises a lip extending in a radial direction.

18. The implantable pulse generator according to claim 17, wherein the annular shoulder of the substantially cylindrical seal member comprises a lip channel configured to engage with the lip.

19. The implantable pulse generator according to claim 14, wherein the connector member comprises a circumferential groove defined on an outer surface of the substantially cylindrical outer body.

20. The implantable pulse generator according to claim 19, wherein the circumferential groove is configured to engage with a lead wire of a stimulation lead or a corresponding projection of a header of the implantable pulse generator.
